(19) United States
(12) Patent Application Publication
(10) Pub. No.: US 2012/0100552 A1
(43) Pub. Date: Apr. 26, 2012

(54) Microfluidic Liquid Heating Method And Apparatus

(76) Inventors: Richard Welle, Huntington Beach, CA (US); Kyle Chrystal, Los Angeles, CA (US)

(21) Appl. No.: 13/277,137

(22) Filed: Oct. 19, 2011

Related U.S. Application Data
(60) Provisional application No. 61/455,518, filed on Oct. 20, 2010.

(51) Int. Cl. C12Q 1/68; C12M 1/00

(52) U.S. Cl. 435/6.12; 435/289.1

(57) ABSTRACT

Systems and methods for avoiding problems due to over-pressurization in a closed microfluidic device when the contained fluid is heated, by providing a well-defined volume for expansion of the liquid contents during heating, such as in one embodiment filling the expansion volume with air, and in other embodiments, filling the expansion volume with vapor-phase fluid from the liquid in the microfluidic device before the device is sealed and in yet other embodiments providing structures to maintain uniform temperatures over the entire closed volume in the microfluidic device.

### CROSS REFERENCE TO RELATED APPLICATION

This application claims the benefit of U.S. 61/455,518 filed on Oct. 20, 2010, and which is incorporated by reference herein.

### FIELD OF INVENTION

The invention relates generally to systems and methods for processing fluids in microfluidic devices. More specifically, this invention relates to systems and methods for heating fluids in closed microfluidic devices.

### BACKGROUND

Microfluidic devices, incorporating sub-millimeter flow channels, can be used to perform various biomedical analyses, and are useful for a broad range of additional applications. These devices are often designed in a manner analogous to printed circuit boards in that the flow channels are configured in planar patterns in either one or multiple layers. The devices are often then built by laminating various layers of material together such that they enclose the flow channels and other features, such as fluid storage reservoirs, reaction volumes, and incubation cells, in the interfaces between the layers. As such, the fluid-filled features in the microfluidic device often have very large aspect ratios, with the depth (in a direction perpendicular to the plane containing the features) being much less than the length or width.

In order to carry out biomedical analyses, as well as many other functions, it is often necessary to heat the fluids contained in the microfluidic device. For example, to carry out a polymerase chain reaction (PCR), the sample must be heated and cooled cyclically between about 55 and 95 C many times. At 95 C, water has a vapor pressure nearly equal to atmospheric pressure. To prevent the formation of vapor bubbles that might force some of the sample out of the heated volume, all inlets and outlets to/from the volume are preferably closed by valves or other seals.

In microfluidic devices, the flow of fluid is often dominated by surface tension effects, and great care is normally taken to avoid the introduction of bubbles in the flow path since surface tension effects, combined with the small size and closed paths, limit the options for removal of bubbles. It is also important to avoid bubbles because trapped bubbles can be a significant fraction of the total volume of liquid in the system, and this could lead to uncertainties in interpretation of results or analyses that depend on knowing the volumes of the fluids involved.

Thus, when performing many kinds of biomedical analyses in microfluidic devices, the fluid being heated is preferably completely enclosed in a reservoir with a fixed volume, and the volume does not contain bubbles. If an analysis requires heating, the heated fluid will tend to expand according to the coefficient of expansion of the fluid. For water (a typical fluid used in biomedical analyses), the coefficient of expansion is such that the water will expand almost 4% in volume on heating from 25 C (room temperature) to 95 C (as required for PCR). If the water is enclosed in a reservoir that has a rigidly fixed volume, then, due to the expansion of the water, the pressure in the reservoir could be expected to increase well beyond the increase in vapor pressure of the water, perhaps to a pressure of hundreds of atmospheres, depending on the rigidity of the reservoir walls. This phenomenon would be offset somewhat by thermal expansion in the materials used to fabricate the reservoir, which would lead to a volume increase in the reservoir corresponding to the volume increase of the fluid. However, most plastics typically used in the manufacture of microfluidic devices have volumetric expansion coefficients less than half that of water, and glass, which is also used in many microfluidic devices, has a volumetric expansion coefficient less than 1/10 that of water.

Thus, an increase in temperature will lead to the water expanding more than the container, causing an increase in pressure in the system or, more likely, a distortion of the reservoir walls to compensate for the expansion of the water. This distortion can lead to cracking and/or delamination of the reservoir walls, which can lead, in turn, to leaks in the system.

To avoid this problem, allowance must be made for the expansion of the liquid in the reservoir if it is to be heated. In the past, this allowance for expansion has often been provided (if unintentionally) either by trapped bubbles or by leaky valves enclosing the volume. As the technology of microfluidics has improved, it has become easier to enclose bubble-free volumes of liquid between leak-tight valves.

Thus, there exists a need for a microfluidic device with a heating chamber that makes allowance for thermal expansion of the fluid contained therein.

There further exists a need for a microfluidic device with a heating chamber that makes allowance for thermal expansion of the fluid contained therein in a manner that will not lead to uncertainties in the volume of fluid contained in the heating chamber.

There further exists a need for a microfluidic device with a heating chamber that makes allowance for thermal expansion of the fluid contained therein without leading to significant pressure increases in the fluid in the heating chamber.

### SUMMARY

The microfluidic devices and methods according to the present invention overcome the drawbacks of known devices and methods by providing a means for heating a fluid in a closed device without leakage or generation of excessive pressures.

These and other embodiments, features, aspects, and advantages of the invention will become better understood with regard to the following description, appended claims and accompanying drawings.

### BRIEF DESCRIPTION OF THE DRAWINGS

The foregoing aspects and the attendant advantages of the present invention will become more readily appreciated by reference to the following detailed description, when taken in conjunction with the accompanying drawings, wherein:

FIG. 1 is a diagram illustrating a prior art microfluidic system with a fluid reservoir, a heater, and inlet and outlet channels and valves.

FIG. 2 is a diagram illustrating a microfluidic system with a fluid reservoir, a heater, inlet and outlet channels and valves, and an expansion reservoir for expansion of the fluid.

FIG. 3 is a diagram illustrating a microfluidic system with a fluid reservoir, a heater, inlet and outlet channels and valves, and an elongated expansion reservoir for expansion of the fluid.

FIG. 4 is a diagram illustrating a microfluidic system with a first fluid reservoir, a first heater, inlet and outlet channels and valves, a second reservoir in line with the first for expansion of the fluid, and a second heater for the second reservoir.

FIG. 5 is a diagram illustrating a microfluidic system with a first fluid reservoir, a first heater, inlet and outlet channels and valves, a second reservoir in line with the first for expansion of the fluid, a second heater for the second reservoir, and an elongated channel connecting the first reservoir with the second reservoir.

FIG. 6 is a diagram illustrating a microfluidic system with a first fluid reservoir, a first heater, inlet and outlet channels and valves, a second reservoir in line with the first for expansion of the fluid, a second heater for the second reservoir, and an elongated channel connecting the first reservoir with the second reservoir, the first heater being configured to heat both the first reservoir and the elongated channel.

FIG. 7 is a diagram illustrating a microfluidic system with a fluid reservoir, a heater, inlet and outlet channels and valves, and a second heater in thermal communication with only a portion of the fluid reservoir.

FIG. 8 is an enlarged view diagram illustrating a portion of the microfluidic system of FIG. 7, further including bubble nucleation sources in the wall of the fluid reservoir.

FIG. 9 is a diagram illustrating a microfluidic system with a fluid reservoir, inlet and outlet channels and valves, an elongated expansion reservoir for expansion of the fluid, and a heater configured to heat both the fluid reservoir and the elongated expansion reservoir.

Reference symbols or names are used in the Figures to indicate certain components, aspects or features shown therein. Reference symbols common to more than one Figure indicate like components, aspects or features shown therein.

### DETAILED DESCRIPTION

In accordance with embodiments described herein, the present microfluidic system is applicable to any microfluidic process in which a volume of fluid must be tightly enclosed and then heated, and in which it is desired to avoid excessive pressure increases. A specific example of such a system would be the polymerase chain reaction (PCR), where it is necessary to heat a biological sample to temperatures near 95 C without loss of fluid, followed by cooling to about 55 C. This temperature swing is repeated multiple times for a specific microfluidic system. While this is an immediate example of an application for the present invention, it should be understood that the present invention can be applied to any microfluidic system requiring heating of a fluid in a closed volume.

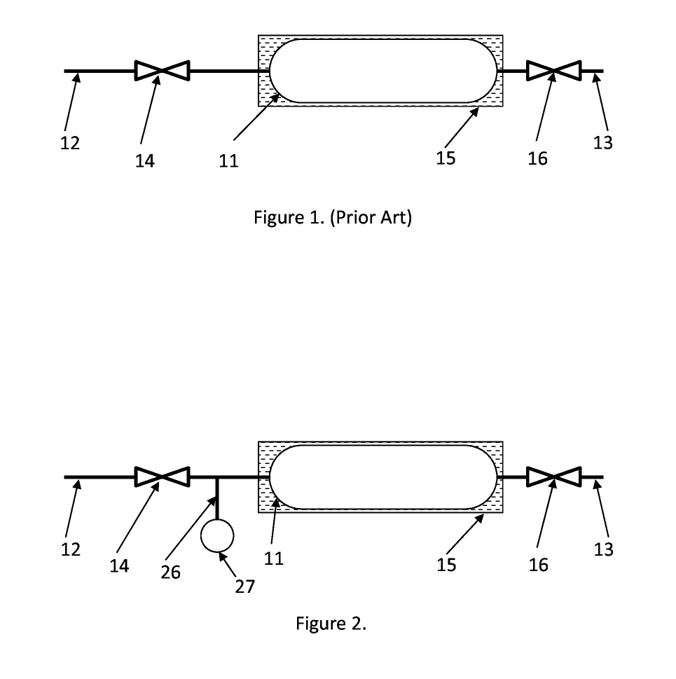

In conducting many biomedical analyses, it is useful to amplify the DNA content of a sample through the use of PCR, a chain reaction that works, in part, by cycling the sample between three different temperatures in a repeated pattern. In order that each analysis be quantitative, it is preferred that the volume of the sample be known with some precision. To accomplish this in a microfluidic system, the sample would preferably be introduced into a container of a known volume, and sealed at an inlet and outlet such that the required volume is between the inlet and outlet seals. FIG. 1 shows the currently-known technology for this application. In this figure, the fluid is introduced through an inlet flow channel 12 through an inlet valve 14 into a reservoir 11. The surface properties of the channels and reservoir are preferably controlled so as to control the wetting of the walls by the fluid and to avoid trapping bubbles. Thus, the fluid flow is continued until the reservoir is filled and fluid has begun to flow out the outlet channel 13. The reservoir 11 is in good thermal contact with a heater 15 that can be used to change the temperature of the reservoir, and the fluid contained therein, on demand. The fluid flow channels between the reservoir and the valves may not be fully in contact with the heater, with the result that the fluid in the flow channels is not subject to the same thermal cycling as the fluid in the reservoir. Thus, the flow channel volumes are preferably kept very small compared to the volume of the reservoir so that the volume of fluid not exposed to the thermal cycling program is a small fraction of the overall volume.

A key factor in providing an accurate quantitative analysis is ensuring that the reservoir, and therefore the entire volume between the valves, is filled, and all bubbles are excluded. This process, although beneficial in providing a well-defined volume of fluid, has a drawback in that there is no room to accommodate thermal expansion in the fluid. While the specific temperatures required for the PCR vary somewhat from one case to another, the peak temperature is typically between 94 and 96 C. For a typical case analysis the sample would start out at room temperature (22 C), where the density of liquid water is 997.8 kg/m³. The sample is heated to 96 C, where the density of liquid water (at one atmosphere pressure) is 961.2 kg/m³. Thus, if the pressure is kept constant at one atmosphere, water undergoing a temperature increase from 22 to 96 C will experience a 3.8% volume increase. With the same temperature increase, a chamber made of polycarbonate will experience a volume increase of only 1.56%, and a chamber made in glass will experience a volume increase of only 0.19%. The expansion of the water on heating will thus lead to an increase in the pressure in the system, possibly causing the chamber to distort, crack, or delaminate.

FIG. 2 shows a microfluidic device similar to that depicted in FIG. 1, but with an added feature to accommodate the expansion of the water on heating. The microfluidic device of FIG. 2 includes all the features of the device shown in FIG. 1, but further includes an expansion chamber 27 connected to the flow line 12 through an expansion flow channel 26. In FIG. 2, the expansion flow channel 26 is shown connecting to the main flow system through the inlet flow channel 12. It would be functionally equivalent to have the expansion flow channel 26 connect to the main flow system through either the outlet flow channel 13 or directly to the main reservoir 11, as long as the point of connection is between the inlet 14 and outlet 16 valves. In this device, when fluid is introduced through the inlet 12, it will flow into the main chamber 11 and fill it as before. Because the expansion chamber is attached to the main flow path through a single line, there is no vent path on the expansion chamber. Thus, as the main chamber is filled with fluid, air will be trapped in the expansion chamber, preventing the flow of liquid into the expansion chamber during the fill process. Assuming the pressure is maintained approximately constant during the fill process, the liquid should not travel significantly into the expansion channel, and the volume of liquid in the system should be essentially the same as the volume of the main reservoir. When the valves are closed and the liquid in the main reservoir is heated with the heater 15, the liquid will expand into the expansion channel 26 and into the expansion reservoir 27. This will prevent a significant buildup of pressure in the main reservoir. There will be a small increase in pressure that depends on the volume of the expansion reservoir. This comes about because the expansion reservoir is closed and the expansion of the liquid into the expansion reservoir will compress the air in the expansion reservoir. If the expansion reservoir is relatively large compared to the total expansion volume of the liquid, then the pressure increase will be relatively small.

One possible issue that may arise in using the microfluidic device of FIG. 2 comes about during the cooling portion of the cycle. Specifically, if the liquid in the main reservoir is heated sufficiently that a significant volume of liquid flows into the expansion reservoir, then, during the cooling phase, when the liquid contracts to its original volume, liquid will be withdrawn from the expansion channel 26. Preferably, this would also result in the liquid being withdrawn from the expansion reservoir 27. However, it is possible that the liquid/air interface in the expansion reservoir would deform during the withdrawal process in such a way as to allow air into the expansion channel 26, and thence into the main reservoir 11, while leaving some of the liquid behind in the expansion volume 27.

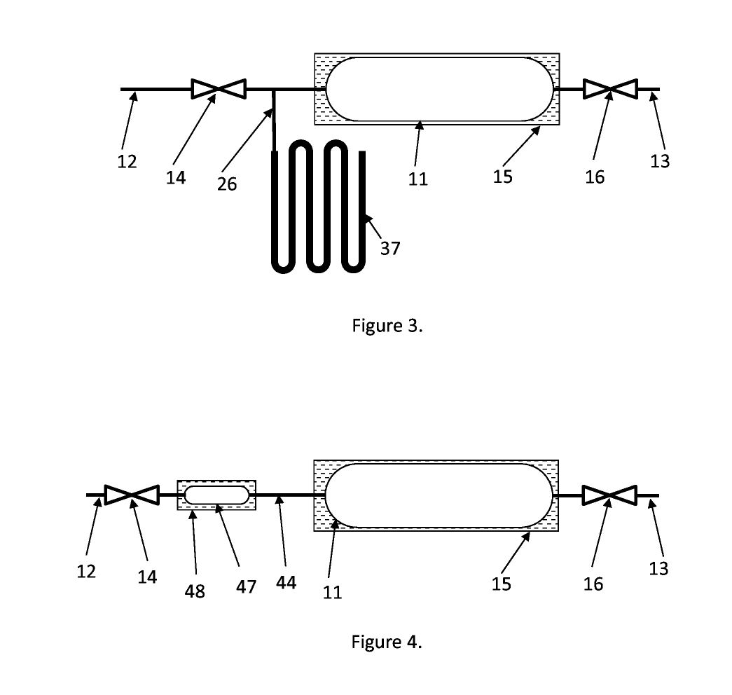

There are various ways to avoid this problem. FIG. 3 illustrates one embodiment of the present invention that limits the probability of air being drawn into the main reservoir 11. In this configuration, the expansion volume 37 takes the shape of an elongated microfluidic channel, preferably with hydrophobic walls. In such a channel, surface tension will keep the air/liquid interface approximately normal to the axis of the channel. Thus, liquid flowing into the expansion volume 37 will behave much as the liquid rising in a thermometer and will maintain a single liquid/air interface that does not allow for the transport of air bubbles out of the expansion volume 37 until all the liquid has been withdrawn. As in the example of FIG. 2, as the liquid is first introduced into the microfluidic system, the expansion channel 26 and expansion volume 37 have only a single inlet and no vent. Thus, little liquid flows into the expansion channel as the main reservoir 11 is filled. After the valves are closed and the liquid in the main reservoir is heated, it will expand and flow into the expansion channel 26 and thence into the expansion volume 37, always maintaining a single liquid/air interface approximately normal to the axis of the channel. When the heater 15 is switched off and the liquid in the main reservoir 11 cools and contracts, liquid is withdrawn from the expansion volume 37 though the expansion channel 26. In order to prevent over pressurization of the microfluidic system, the expansion volume must be large enough to receive the fluid expanding out of the main reservoir 11. It would be possible to have the distant end of the expansion volume 37 open to the atmosphere, but this could have unintended consequences if the pressure in the microfluidic system is not well controlled with respect to atmospheric pressure, so it is preferable that the distant end of the expansion volume 37 be closed.

Another embodiment of the present invention that limits the probability of air being drawn into the main reservoir is illustrated in FIG. 4. In this configuration the expansion volume 47 is in direct line with the flow channel 44 between the inlet valve 14 and the main reservoir 11, and is in thermal contact with a heating element 48. In this description, it is assumed that the microfluidic device is filled through the inlet 12 and that the fluid flows through the expansion reservoir before reaching the main reservoir. This configuration was chosen for convenience and clarity of the description. It should be understood that the inlet and outlet could be reversed, such that the fluid flows first through the main reservoir before reaching the expansion reservoir, without affecting the functionality of the device as described herein. In this configuration, when the liquid fills the microfluidic device, the expansion reservoir will be filled with liquid before the main reservoir. Once both reservoirs and all flow paths are filled with liquid, all air has been excluded from the microfluidic device. To use this device in a manner that allows for expansion of fluid during heating in the main reservoir, it is first necessary to expel the liquid from the expansion reservoir. This is done by heating the expansion reservoir 47 using the corresponding heater 48, until the temperature in the expansion reservoir exceeds the boiling point of the liquid in the system. This is done with one or both of the inlet 14 and outlet 16 valves open so that the liquid in the expansion reservoir can vaporize without increasing the pressure in the system. After the expansion reservoir is full of vapor, the valves are closed to seal the system. Then, without cooling the expansion reservoir, the main reservoir is heated as necessary for the specified process. Heating the main reservoir will cause the liquid therein to expand, forcing some liquid through the flow channel 44 into the expansion reservoir 47. As this liquid flows into the expansion reservoir, it will be heated to the expansion reservoir temperature, which is above the boiling point, and the liquid will therefore evaporate, adding to the vapor in the expansion reservoir. This additional vapor will cause the pressure to rise, but only to the pressure corresponding to the vapor pressure of the liquid at the temperature of the expansion reservoir. For example, assuming the liquid in the microfluidic device is water and the initial pressure in the system is one atmosphere (101 kPa), the expansion reservoir would have to have been heated to at least 100 C to vaporize the water therein. To be certain of eliminating all liquid water from the expansion reservoir, the temperature may preferably be increased to a higher value, for example 105 C, before sealing the microfluidic system by closing the valves. When the valves are closed, the expansion volume will contain water vapor at one atmosphere pressure and a temperature of 105 C. When the main reservoir is heated and the water therein expands, some additional water will be forced into the expansion reservoir where it will evaporate. The additional water vapor will cause the pressure in the system to increase. However, when the system pressure reaches the vapor pressure of water at the 105 C temperature of the expansion volume (121 kPa), no further evaporation will take place. Further expansion of the water in the main reservoir will continue to force more water into the expansion reservoir, but no further evaporation will take place since the pressure in the system is now equal to the vapor pressure at the expansion reservoir temperature.

As noted, the expansion reservoir 47 is filled with vapor by heating it to a temperature above the boiling point of the liquid therein at the local pressure. Once the expansion reservoir is filled with vapor and the valves 14 and 16 are closed, an alternative operating procedure would allow the temperature of the expansion reservoir to be cooled to some intermediate temperature that is below the boiling point of the liquid, but above the maximum expected temperature of the main reservoir. For example, for conducting PCR, the main reservoir would be expected to reach a maximum temperature of 96 C. Thus, once the microfluidic device of FIG. 4 is filled with liquid, the heater 48 is used to raise the temperature of the expansion reservoir 47 to 105 C to expel all the liquid therein and replace it with water vapor. The valves 14 and 16 are then closed, sealing the system. The power to the heater 48 could then be reduced, reducing the temperature of the expansion reservoir 47 to 100 C, for example, and then maintaining it there through the thermal cycling of the main reservoir 11. Since the temperature of the main reservoir is always less than the temperature of the expansion reservoir, the vapor pressure in the expansion reservoir will be higher, which will inhibit formation of vapor bubbles in the main reservoir, but will allow expansion of the liquid from the main reservoir into the expansion reservoir.

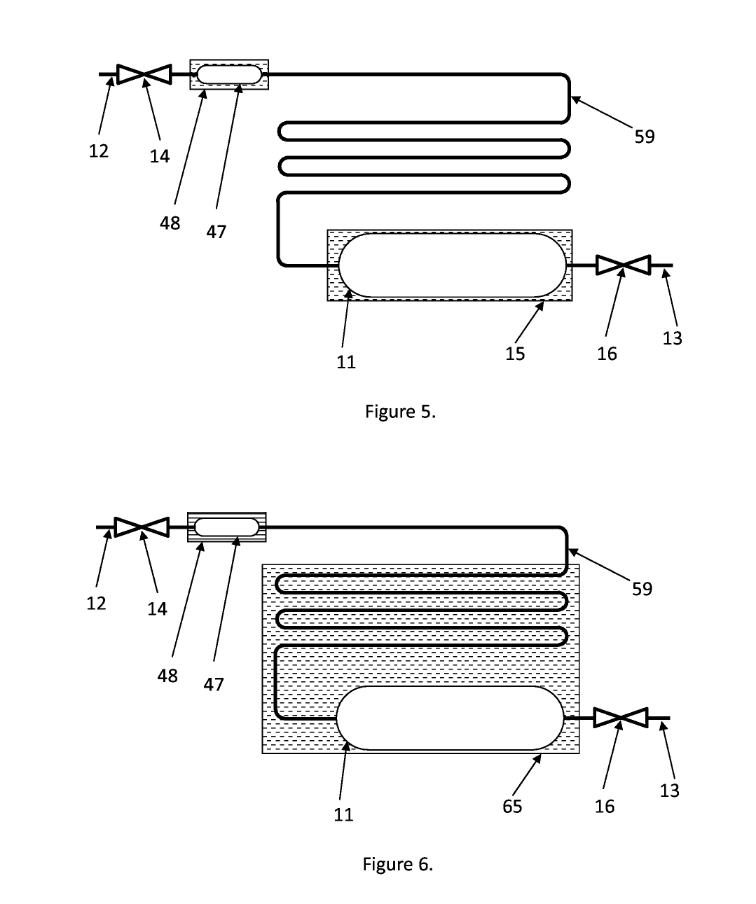

One possible side effect of thermal cycling in the microfluidic device of FIG. 4 is that periodic cycling of the temperature of the main chamber will result in a periodic flow of fluid into and out of the expansion reservoir, which can lead, in turn, to cyclic phase changes in the fluid moving into and out of the expansion reservoir. This cyclic phase change can lead to an inadvertent distillation process where dissolved components in the fluid could be selectively deposited in the expansion reservoir, while the fluid moving back and forth in the flow channel 44 connecting the two reservoirs could become depleted in dissolved components. If the expansion is great enough and the connecting channel 44 short enough, then the fluid depleted in dissolved components in the expansion reservoir during the hot part of the cycle could mix with the fluid remaining in the main reservoir during the cold part of the cycle. This issue can be avoided by extending the length of the connecting channel as illustrated in FIG. 5. In this figure, the channel 59 connecting the expansion chamber 47 to the main thermal cycling chamber 11 has a microfluidic cross section such that flow through the channel is laminar to prevent turbulent mixing of the fluid as it flows through the channel. The channel length is selected to be long enough that the total volume of the channel is greater than the anticipated volume of the expansion of the fluid in the main chamber during the thermal cycling. For example, if the main chamber has a volume of 100 microliters and is filled with water, and the anticipated temperature change is from room temperature (22 C) to 96 C, then the density of the water will change from an initial value of 997.8 kg/m³ to a minimum value of 961.2 kg/m³, and the initial volume of 100 microliters of water will expand to 103.8 microliters. Thus, the net volume change of the liquid will be 3.8 microliters. A typical microfluidic channel might be 100 microns deep and 500 microns wide, so the length of channel required to accommodate the full expansion of fluid from the main reservoir would be 76 mm. As an alternative, the channel could be 0.5 mm wide and 0.5 mm deep, in which case the channel would need to be only 15 mm long to accommodate the full expansion of fluid from the main reservoir. These channel dimensions are representative of possible sizes, and it should be understood that this method will work with any channels small enough that the flow is laminar.

In the configuration of FIG. 5, the fluid in the flow channel 59 is not subjected to thermal cycling in the same manner as the fluid in the reservoir 11. When the fluid in the flow channel returns to the reservoir during the cooling phase, there can be some mixing in the reservoir of fluids that have and have not been subjected to the preferred thermal cycling. This concern can be avoided, as illustrated in FIG. 6, by extending the heater 65 to heat a portion of the flow channel with a volume at least as large as the expected expansion volume of the fluid in the reservoir. Thus, while fluid will move back and forth between the expansion reservoir and the heated section of the flow channel, there will be a boundary between the fluid always in the heated section of the flow channel and the fluid that moves between the heated and non-heated sections of the flow channel. That boundary will move back and forth along the heated portion of the flow channel, moving away from the main reservoir 11 as it is heated, and moving toward the main reservoir as it is cooled. However, if the volume of the heated section of the flow channel is larger than the total expansion volume of the fluid in the reservoir 11, then the boundary will never cross into the main reservoir 11, and only that portion of the fluid that is subjected to thermal cycling in the flow channel will enter and mix with the fluid in the reservoir 11.

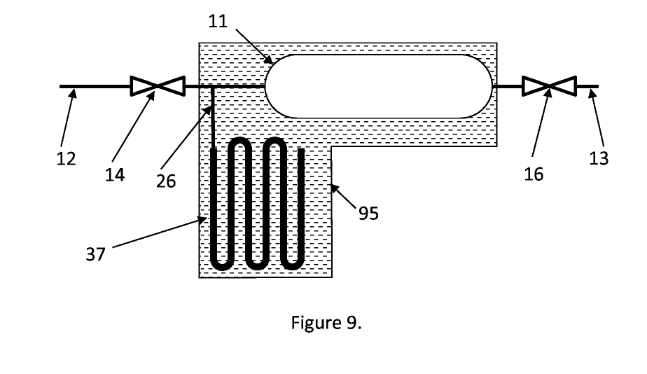

Similarly, in the microfluidic device configured as shown in FIG. 3, fluid expanding into the elongated expansion reservoir 37 will not be heated to the same level as the fluid remaining in the reservoir. This problem can be overcome by extending the heater to include all of the expansion reservoir, as illustrated in FIG. 9. In FIG. 9 the expansion reservoir 37 is in thermal contact with the heater 95 and experiences the same temperature cycles as the reservoir 11.

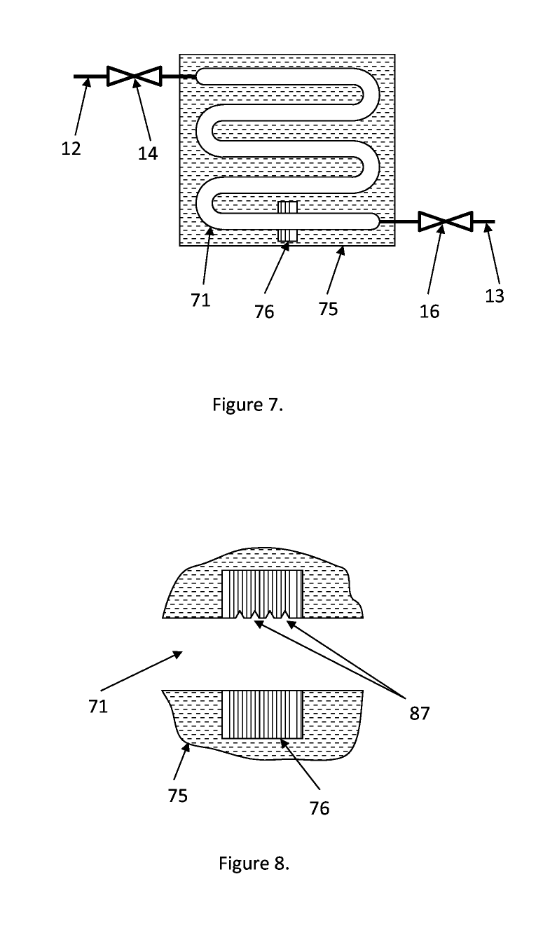

Another embodiment of the invention is illustrated in FIG. 7. In this embodiment, the expansion volume is not separated from the main thermal cycling reservoir, but is instead provided within the thermal cycling reservoir through the use of an additional heating step. Thus, the thermal cycling reservoir 71 is in thermal contact with a primary heater 75 that provides the heating necessary to control the temperature of the fluid in the thermal cycling reservoir as desired. In addition, a small portion of the main thermal cycling reservoir 71 is in thermal contact with an additional heater (or additional segment of the primary heater) 76 that is used to create the expansion volume. The size of the additional heater 76 is such that the fractional area of the reservoir 71 exposed to the additional heater 76 is at least equal to the fractional change in density expected in the liquid over the temperature range used for the heating process. In operation, the liquid flows into the thermal cycling reservoir 71 through inlet channel 12 and inlet valve 14. When the reservoir 71 is full up to the outlet valve 16, the flow is stopped. With one or both of the inlet 14 and outlet 16 valves open, the additional heater 76 is used to heat the adjacent segment of the thermal cycling reservoir 71 to a temperature above the boiling point of the liquid in the reservoir, converting the liquid to vapor in that portion of the reservoir. Then, with the reservoir containing both liquid and the small vapor bubble, both valves 14 and 16 are closed, sealing the liquid and vapor between the valves. The additional heater 76 can then be turned off. Assuming the reservoir is leak tight, the pressure in the reservoir will drop to the vapor pressure of the fluid at the ambient temperature but, there being no source for additional liquid or air, a vapor bubble (at the lower pressure) will remain in the reservoir. From this condition, thermal cycling can commence throughout the full reservoir. The liquid in the reservoir will expand, filling the vapor bubble with each heating cycle, but reforming the vapor bubble on each cooling cycle. However, if the vapor bubble is sufficiently large at the time the valves are closed to seal the device, then the internal pressure will never exceed the pressure initially present at the time the valves were closed.

Reliable operation of the device illustrated in FIG. 7 requires the formation of a bubble of about the same size as the additional heater 76. One might expect that, under equilibrium conditions, simply heating that portion of the reservoir to a temperature above the local boiling point would lead to vaporization of sufficient fluid to form a bubble of the appropriate size. However, bubble formation in a clean, smooth-walled container can involve non-equilibrium processes. Specifically, it is entirely possible to superheat a liquid when there are no bubble nucleation sources present. Although rare in everyday experience, superheating is relatively easy to achieve (even if unintentionally) in microfluidic devices. If the liquid is superheated, it is possible that no vapor phase would form, even at temperatures well above the normal boiling point of the liquid. If this happens, and the valves are closed without the presence of a vapor bubble, then the pressure in the system during thermal cycling would greatly exceed atmospheric pressure. One way to avoid this problem is to increase the temperature of the additional heater 76 enough that superheating is overcome, forcing the formation of a bubble. While potentially a useful approach, there are a couple of drawbacks to this idea. First, if the walls of the reservoir are sufficiently clean and smooth, it may require several tens of degrees of additional heating to ensure the formation of a bubble in the heated region. Particularly if the reservoir is made of polymeric materials, the temperature required may exceed the maximum working temperature of the materials. The second problem is that in cases of significant superheating, once vapor formation commences, bubble formation can be explosive in nature, whereby a large fraction of the superheated liquid is converted to vapor in a very rapid process. This can lead to over pressurization of the microfluidic device, and/or can lead to the liquid being expelled from much more of the thermal cycling reservoir 11 than just the area adjacent to the additional heater 76, leaving less than the desired quantity of liquid in the reservoir.

To avoid this problem, the area of the thermal cycling reservoir adjacent to the additional heater 76 can be provided with nucleation sources to promote vapor formation. One possible approach is illustrated in FIG. 8, which shows a close-up of the region of the reservoir around the additional heater 76. Normally, in microfluidic devices, the preference is to keep the walls as smooth as possible to avoid trapping of bubbles. However, microscopic bubbles make excellent nucleation sources for bubble formation in boiling processes. Thus, as illustrated in FIG. 8, the wall of the thermal cycling reservoir 71 includes one or more small indentations 87 intended to capture a small volume of air during the liquid filling process. These trapped bubbles are small enough that they do not significantly affect the overall volume of the liquid in the reservoir 71, but the air/liquid interface at the bubble surface provides a surface for vapor formation and allows for the orderly growth of a bubble in the reservoir during the heating with the additional heater 76. Each of the previously described configurations of the expansion volume that depend on vapor formation can also benefit from the presence of nucleation sources to trigger orderly bubble growth.

Although specific embodiments of the invention have been described, various modifications, alterations, alternative constructions, and equivalents are also encompassed within the scope of the invention.

The specification and drawings are, accordingly, to be regarded in an illustrative rather than a restrictive sense. It will, however, be evident that additions, subtractions, deletions, and other modifications and changes may be made thereunto without departing from the broader spirit and scope of the invention as set forth in the claims.

What is claimed is:

1. A microfluidic device comprising:
a first fluid reservoir in thermal communication with a heat source;
an inlet flow channel in fluid communication with said first fluid reservoir;
an inlet valve adapted to open and to close said inlet flow channel;
an outlet flow channel in fluid communication with said first fluid reservoir;
an outlet valve adapted to open and to close said outlet flow channel;
a single microfluidic connecting flow channel;
a second fluid reservoir in fluid communication with said first fluid reservoir and with said single microfluidic connecting flow channel; and,
said second reservoir being unvented.

2. The microfluidic device of claim 1 wherein said second fluid reservoir has a volume between 2 and 10 percent of the volume of said first fluid reservoir.

3. The microfluidic device of claim 1 wherein said second fluid reservoir is in the shape of an elongated channel having a cross sectional area less than 1 mm square.

4. A microfluidic device comprising:
a first fluid reservoir in thermal communication with a first heat source;
a second fluid reservoir in thermal communication with a second heat source;
a first microfluidic channel in fluid communication with said first fluid reservoir;
a second microfluidic channel in fluid communication with said second fluid reservoir;
a first valve adapted to open and to close said first microfluidic channel;
a second valve adapted to open and to close said second microfluidic channel; and,
a third microfluidic channel in fluid communication with said first fluid reservoir and with said second fluid reservoir.

5. The microfluidic device of claim 4 wherein said third microfluidic channel has a cross sectional area not greater than 1 square mm and a length sufficient to provide a total volume not less than 2 percent of the volume of said first fluid reservoir.

6. The microfluidic device of claim 5 wherein said first heat source is in thermal communication with both said first fluid reservoir and said third connecting microfluidic channel.

7. A microfluidic device comprising:
a fluid reservoir having a predesignated volume and in thermal communication with a first heat source;
an inlet flow channel in fluid communication with said fluid reservoir;
an inlet valve adapted to open and to close said inlet flow channel;
an outlet flow channel in fluid communication with said fluid reservoir;
an outlet valve adapted to open and to close said outlet flow channel; and,
a second heat source in thermal communication with a selected fraction of said fluid reservoir, said selected fraction having between 1 percent and 10 percent of the total volume of said fluid reservoir.

8. The microfluidic device of claim 7 wherein said fluid reservoir includes at least one bubble nucleation source positioned within said selected fraction.

9. The microfluidic device of claim 4 wherein said second fluid reservoir includes at least one bubble nucleation source.

10. A method of heating a liquid in a sealed microfluidic device comprising:
introducing at a first temperature and at a first pressure a liquid into a microfluidic device having a predetermined volume, said liquid having a vapor pressure at said first temperature less than said first pressure;
heating a portion of said liquid to a second temperature, said second temperature above the boiling point of said liquid at said first pressure, to create a vapor bubble having a bubble volume at least as large as the anticipated expansion volume of said liquid at a third temperature, said third temperature intermediate between said first temperature and said second temperature and to leave a residual liquid portion;
sealing said residual liquid portion and said vapor bubble together in said predetermined volume in said microfluidic device; and,
heating said residual liquid portion to said third temperature and allowing said residual liquid portion to expand into space occupied by said vapor bubble.

11. The microfluidic device of claim 1 wherein both first and second fluid reservoirs are in thermal communication with said heat source.
